(19) United States
(12) Patent Application Publication
(10) Pub. No.: US 2011/0097302 A1
(43) Pub. Date: Apr. 28, 2011

(54) IL-1RA-POLYMER CONJUGATES

(76) Inventors: Ta Tung Yuan, Taipei (TW); Shih-Hsien Chuang, Taipei (TW); Tzu-Yin Lin, Taishan Township (TW)

(21) Appl. No.: 12/837,789

(22) Filed: Jul. 16, 2010

Related U.S. Application Data
(60) Provisional application No. 61/226,168, filed on Jul. 16, 2009.

(51) Int. Cl. A61K 38/20; C07K 17/08; A61P 29/00; A61P 37/02

(52) U.S. Cl. 424/85.2; 530/351

(57) ABSTRACT

This invention relates to protein-polymer conjugates described in the specification. Also disclosed are a method for preparing a protein-polymer conjugate and using such a conjugate in treating various immune disorders.

### RELATED APPLICATION

This application claims priority of U.S. Provisional Application No. 61/226,168, filed on Jul. 16, 2009. The prior application is incorporated by reference in its entirety.

### BACKGROUND

Advance in cell biology and recombinant protein technologies has led to the development of protein therapeutics. Yet, many protein therapeutics, such as IL-1 receptor antagonist (IL-1ra), are susceptible to proteolytic degradation and therefore have short half-lives in the circulating system. Other disadvantages include low bioactivity. There is a need for effective protein therapeutics that have prolonged half lives and satisfactory bioactivity.

### SUMMARY

This invention is based on a discovery of polymer-IL-1ra conjugates that have long half lives in the human blood (e.g., longer than 12 hours, 48 hours, or 72 hours), while maintaining the protein activities.

An aspect of the present invention relates to a conjugate including (1) an IL-1ra moiety, (2) a spacer that is covalently bonded to the IL-1ra moiety by a thio-ether bond, and (3) a polymer moiety that is covalently bonded to the spacer, the spacer being a hydrocarbon moiety containing 1-20 carbon atoms and 1-10 heteroatoms, and the polymer moiety having a molecular weight of about 5-100 kilodaltons or kD (e.g., 25-90 kD and 30-80 kD). The conjugate may have more than 1 polymer moiety (e.g., 2-5 polymer moieties).

The term “spacer” refers to a multi-valent (e.g., bi-valent or tri-valent) C₁₋₂₀ hydrocarbon group that bonds to both the polymer moiety and the protein moiety. The spacer may have one or more functional groups substituted or inserted in the hydrocarbon backbone. Examples of functional groups include, but are not limited to, —O—, —S—, carboxylic ester, carbonyl, carbonate, amide, carbamate, urea, sulfonyl, sulfinyl, amino, imino, hydroxyamino, phosphonate, or phosphate group.

The term “polymer moiety” refers to a mono-valent radical derive from a linear, branched, or star-shaped linear or branched polymer or copolymer. An example of the polymer is polyalkylene oxide, such as polyethylene oxide, polyethylene glycol, polyisopropylene oxide, polybutenylene oxide, and copolymers thereof. The polyalkylene oxide moiety is either substituted or unsubstituted. For example, it can be methoxy-capped polyethylene glycol (mPEG). Other polymers such as dextran, polyvinyl alcohols, polyacrylamides, or carbohydrate-based polymers can also be used to replace polyalkylene oxide, as long as they are not antigenic, toxic, or eliciting immune response.

The term “IL-1ra” refers to human IL-1ra and mutants or variants derived from it that maintains its biological functions. Show below is the sequence of human IL-1ra (SEQ ID NO: 1):

MRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKR
FAFIRSDSGPTTSFESAACPGWFLCTAMEADQPVSLTNMPDEGVMVTKF
YFQEDE

In one embodiment, the polymer-protein conjugate has a formula shown below:

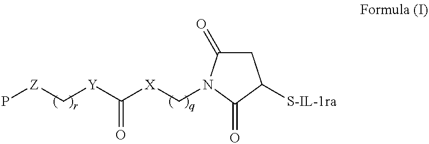

wherein each of X, Y, and Z, independently, is O, NH, or deleted; S-IL-1ra is IL-1ra protein, a sulfur atom of which is linked to the succinimidyl ring in Formula (I); P is a linear or branched polymer moiety; and each of r and q, independently, is 0, 1, 2, 3, 4, or 5.

Referring to Formula (I), a subset of conjugates may have one or more of the following features: q is 2, r is 3, X is deleted and Y is O or NH, X is O or NH and Y is deleted, Z is deleted or O, and P has a molar mass of 5-40 kD or 20-60 kD.

An example of the conjugate is shown below:

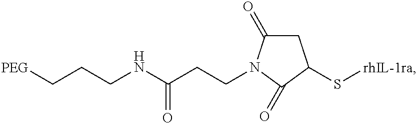

in which the PEG moiety has a molar mass of about 30 kD or about 40 kD. As another example, the conjugate has the following structure:

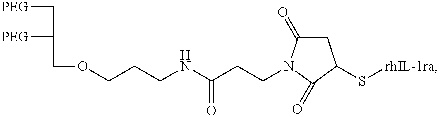

wherein the PEG has a molar mass of about 40 kD.

The protein-polymer conjugate described above can be in the free form or in the form of salt, if applicable. A salt, for example, can be formed between an anion and a positively charged group (e.g., amino) on a protein-polymer conjugate of this invention. Suitable anions include chloride, bromide, iodide, sulfate, nitrate, phosphate, citrate, methanesulfonate, trifluoroacetate, and acetate. Likewise, a salt can also be formed between a cation and a negatively charged group (e.g., carboxylate) on a protein-polymer conjugate of this invention. Suitable cations include sodium ion, potassium ion, magnesium ion, calcium ion, and an ammonium cation such as tetramethylammonium ion.

Another aspect of this invention relates to a method of treating an immune disorder. The method includes administering to a subject in need thereof an effective amount of the just-mentioned conjugate. Examples of the immune disorder include acute and chronic inflammation, diabetes mellitus (including type I and type II diabetes), arthritis (including rheumatoid arthritis, juvenile rheumatoid arthritis, osteoarthritis, and psoriatic arthritis), ankylosing spondylitis, multiple sclerosis, encephalomyelitis, myasthenia gravis, systemic lupus erythematosis, autoimmune thyroiditis, dermatitis (including atopic dermatitis and eczematous dermatitis), dermatomyositis, polymyositis, psoriasis (e.g., plaque psoriasis), Sjögren's Syndrome, Crohn's disease, aphthous ulcer, iritis, conjunctivitis, keratoconjunctivitis, inflammatory bowel diseases, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, graft-versus-host disease, cases of transplantation (including transplantation using allogeneic or xenogeneic tissues) such as bone marrow transplantation, liver transplantation, or the transplantation of any organ or tissue, allergies such as atopic allergy, AIDS, T cell neoplasms such as leukemia or lymphomas, acute hepatitis, angiogenesis related diseases (such as rheumatoid arthritis and cancer), and cardiovascular diseases.

Also within the scope of this invention is a composition containing the conjugate for use in any of the above-mentioned disorders, as well as this therapeutic use and use of the conjugate for the manufacture of a medicament for treating one of these disorders.

The details of one or more embodiments of the invention are set forth in the description below. Other features, objects, and advantages of the invention will be apparent from the description and from the claims.

### BRIEF DESCRIPTION OF DRAWINGS

FIGS. 1(A)-(C) are charts showing HPLC analysis of di-branched m-PEGs: (A) 10 kD, (B) 24 kD, and (C) 40 kD.

FIG. 2 is a chart showing HPLC analysis of DCBpdi007.

FIG. 3 is a chart showing the results that IL-1ra treated with protease inhibitor increased stability of IL-1ra in human serum.

FIG. 4 is a photograph showing the result of PEG conjugation and purification of PEG-IL-1ra.

FIGS. 5(A)-(C) are charts showing results of receptor binding activity of IL-1ra and PEG-IL-1ra with IL-1RI, indicating that the IL-1RI binding activities of DCBpdi005 (B) and DCBpdi007 (C), but not DCBpdi001 and DCBpdi002 (A), were better than that of IL-1ra.

FIG. 6 is a chart showing that PEGylation of IL-1ra increased their stabilities in human serum.

FIGS. 7(A) and 7(B) are charts showing that PEG-IL-1ra, e.g., DCBpdi005 (A) and DCBpdi007 (B), retained the neutralization activity for IL-1β assayed on D10 cells.

### DETAILED DESCRIPTION

The polymer-protein conjugate of this invention contain at least an IL-1ra moiety, a polymer moiety, and a spacer moiety.

IL-1ra is a human protein that acts as a natural inhibitor of IL-1, a cytokine produced by cells of the macrophage/monocyte lineage. It suppresses biological activities caused by IL-1 via binding to IL-1 receptors so as to prevent IL-1 from binding to the same receptors. IL-1 receptor is mostly expressed at inflammatory sites and lymphocytes.

IL-1ra can be used in the conjugate described herein includes human IL-1ra (SEQ ID NO: 1) and its functional equivalents. IL-1ra functional equivalents are polypeptide derivatives of the IL-1ra (SEQ ID NO: 1). They have substantially the activity of IL-1ra, i.e., e.g., binding to IL-1 receptors and preventing IL-1 from binding to the same receptors. IL-1ra and its functional equivalent contains at least one interleukin-1 receptor antagonist domain, which refers to a domain capable of specifically binding to IL-1 receptor family members and preventing activation of cellular receptors to IL-1 and its family members. IL-1 receptor family contains several receptor members. Accordingly, there are several different IL-1 family agonists and antagonists. These IL-1 antagonists may not necessarily bind same IL-1 receptor family members. Here IL-1ra is used to represent all the IL-1 antagonists that bind to IL-receptor family members or/and neutralize activities of IL-1 family members.

An IL-1ra functional equivalent contains an interleukin-1 receptor antagonist domain. This domain refers to a domain capable of specifically binding to IL-1 receptor family members and preventing activation of cellular receptors to IL-1 and its family members. Examples include IL-1ra (U.S. Pat. No. 6,096,728), IL-1 HY1 or IL-1 family member 5 (U.S. Pat. No. 6,541,623), IL-1Hy2 or IL-1 family member 10 (U.S. Pat. No. 6,365,726), IL-1ra beta (U.S. Pat. No. 6,399,573), other IL-1 antagonist members and their functional equivalents, i.e., polypeptides derived from IL-1ra e.g., proteins having one or more point mutations, insertions, deletions, truncations, or combination thereof. They retain substantially the activity of specifically binding to IL-1 receptor and preventing activation of cellular receptors to IL-1. They can contain SEQ ID NO: 1 or a fragment of SEQ ID NO: 1. Preferably, the IL-1ra is a glycosylated mammalian polypeptide. The activity of an Interleukin-1 receptor antagonist may be determined by cell-based IL-1 neutralization assay using IL-1 dependent D10 cells (see Example 2 below), and other IL-1 family member neutralizing assays.

A functional equivalent of SEQ ID NO: 1 refers to a polypeptide derived from SEQ ID NO: 1, e.g., a fusion polypeptide or a polypeptide having one or more point mutations, insertions, deletions, truncations, or a combination thereof. It is at least 70% (e.g., 75%, 80%, 85%, 90%, 95%, 99%, or 100%) identical to SEQ ID NO: 1, and has the above-mentioned conservative interleukin-1 receptor antagonist domain. The variants include biologically active fragments whose sequences differ from the IL-1ra described herein by one or more conservative amino acid substitutions or by one or more non-conservative amino acid substitutions, deletions, or insertions that do not abolish the catalytic activity. All of the functional equivalents have substantially the IL-1ra activity.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) are used.

The amino acid composition of an IL-1ra may vary without disrupting the IL-1ra activity. For example, such a variant can contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a polypeptide is preferably replaced with another amino acid residue from the same side chain family.

The polymer moiety in the conjugate of this invention can be a radical derived from a polymer having a molar mass of 20-100 kD.

The polymer moiety can be a linear mPEG moiety having the following formula:

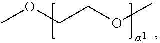

wherein a¹ is selected from the numbers which cause the final molecular weight of the polymeric moiety ranged from 20 kD to 100 kD.

The polymer moiety can also be a branched mPEG moiety having one of the following formulas:

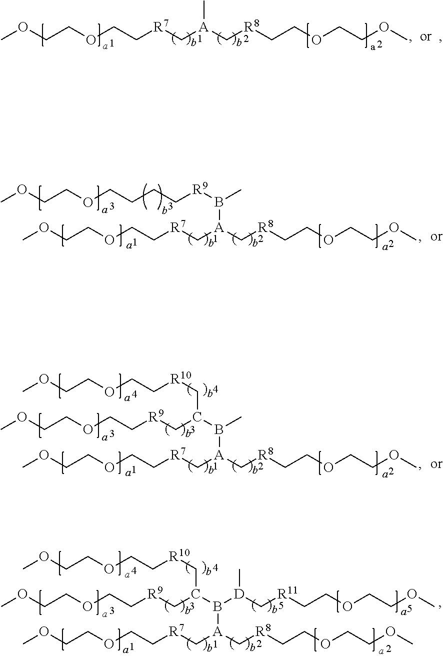

wherein a¹, a², a³, a⁴ and a⁵ are independently selected from the numbers which together cause the final molecular weight of the polymeric moiety ranged from 20 kD to 100 kD; b¹, b², b³, b⁴ and b⁵ are independently selected from 0-6; R⁷, R⁸, R⁹, R¹⁰ and R¹¹ are independently selected from the group consisting of carbonyl, ester, amide, urea, alkoxy, alkyl, alkoxycarbonyl, alkylcarbonyl, and hydroxyalkyl; A, B, C and D are independently selected from the group consisting of C(R¹²)(R¹³), N(R¹²), O and S; and R¹² and R¹³ are independently selected from the group consisting of hydrogen, halogen, alkoxy, alkyl, hydroxyl, alkoxycarbonyl, alkylcarbonyl, and hydroxyalkyl. Examples of branched PEG moieties are shown below:

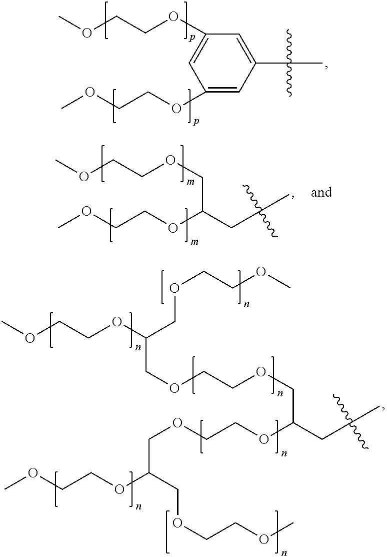

wherein each of p, independently, is 250-700, each m, independently, is 250-1000, and each n, independently, is 50-1000.

The polymer moiety can also be a copolymer mPEG having the following formula:

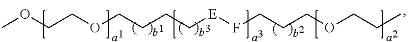

wherein each of a¹, a² and a³ are independently selected from the numbers which cause the final molecular weight of the polymeric moiety ranged from 20 kD to 100 kD; b¹, b² and b³ are independently selected from 0-6; E and F are independently selected from the group consisting of Si(R¹⁴)(R¹⁵) C(R¹⁴)(R¹⁵), N(R¹⁴), O and S; and R¹⁴ and R¹⁵ are independently selected from the group consisting of hydrogen, halogen, alkoxy, alkyl, hydroxyl, alkoxycarbonyl, alkylcarbonyl, and hydroxyalkyl.

The protein-polymer conjugates of the present invention can be prepared by conventional synthetic methods. For example, one can first bond a linker (spacer) molecule to a polymer molecule and subsequently bond IL-1ra to the linker-polymer to form an IL-1ra-linker-polymer conjugate of this invention, or vise versa.

To bond a linker molecule to a polymer molecule, the linker molecule needs to possess a functional group that is reactive to a functional group on the polymer molecule. A reactive group can be a leaving group, a nucleophilic group, or an electrophilic group.

To bond the linker molecule to IL-1ra, the linker molecule needs to possess a functional group (e.g., an electrophilic group) that is reactive to the thiol group of a cysteine residue of the rhIL-1ra (Cys 66, Cys 69, Cys 116, or Cys 122) to form a protein-polymer conjugate of this invention.

The term “leaving group” refers to a functional group that can depart, upon direct displacement or ionization, with the pair of electrons from one of its covalent bonds (see, e.g., F. A. Carey and R. J. Sunberg, Advanced Organic Chemistry, 3rd Ed. Plenum Press, 1990). Examples of a leaving group include, but are not limited to, methansulfonate, triflate, p-tolueesulfonate, iodine, bromide, chloride, trifluoroacetate, succinimidyl (“Su”), p-nitrophenoxy, and pyridine-2-yl-oxy.

The term “nucleophilic group” refers to an electron-rich functional group, which reacts with an electron-receiving group, such as electrophile, by donating an electron pair.

The term “electrophilic group” refers to an electron-poor functional group, which reacts with an electron-donating group, such as a nucleophile, by accepting an electron pair. Michael receptors, containing an α,β-unsaturated ketone moiety or a vinyl sulfone moiety, are a subset of electrophilic groups. They, upon contacting a nucleophile, undergo Michael reaction. Other electrophilic groups include, but are not limited to aldehyde and maleimidyl.

The synthetic methods described above may include steps of adding or removing suitable protecting groups. In addition, synthetic steps may be performed in an alternate sequence or order to give the desired protein-polymer conjugates. Synthetic chemistry transformations, protecting group methodologies (protection and deprotection), and reaction conditions useful in synthesizing applicable protein-polymer conjugates are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

An IL-1ra-polymer conjugate thus synthesized can be further purified by a method such as ion exchange chromatography, gel filtration chromatography, electrophoresis, dialysis, ultrafiltration, or ultracentrifugation.

The protein-polymer conjugate of this invention maintains the activities of rhIL-1ra and has a long half life in the human blood. Thus, this invention also relates to a method of treating a rhIL-1ra-mediated disease, such as immune disease, by administering an effective of the conjugate to a subject in need thereof. Such a subject can be identified by a health care professional based on results from any suitable diagnostic method.

As used herein, the term “treating” or “treatment” is defined as the application or administration of a composition including a protein-polymer conjugate to a subject (human or animal), who has a disorder, a symptom of the disorder, a disease or disorder secondary to the disorder, or a predisposition toward the disorder, with the purpose to cure, alleviate, relieve, remedy, or ameliorate the disorder, the symptom of the disorder, the disease or disorder secondary to the disorder, or the predisposition toward the disorder. “An effective amount” refers to an amount of a protein-polymer conjugate which confers a therapeutic effect on the treated subject. The therapeutic effect may be objective (i.e., measurably by some tests or markers) or subjective (i.e., a subject gives an indication of or feels an effect). Effective doses will vary, as recognized by those skilled in the art, depending on, e.g., the rate of hydrolysis of a protein-polymer conjugate, the types of diseases to be treated, the route of administration, the excipient usage, and the possibility of co-usage with other therapeutic treatment.

To practice the method of the present invention, a composition having one or more of the above-mentioned compounds can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, intraperitoneal, intratracheal or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as TWEENS or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more of the above-described compounds can also be administered in the form of suppositories for rectal administration.

A pharmaceutically acceptable carrier is routinely used with one or more active above-mentioned compounds. The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an above-mentioned compound. Examples of other carriers include colloidal silicon oxide, magnesium stearate, cellulose, sodium lauryl sulfate, and D&C Yellow # 10.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Example 1

### Chemical Synthesis

### (1) Synthesis of the Following IL-1ra Conjugate:

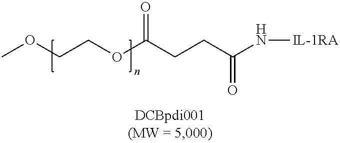

RhIL-1ra (4 mg, 0.26 μmmol) at 1 mg/ml in phosphate buffered saline (PBS, pH 7.5) was mixed with mPEG-succinyl-N-hydroxysuccinimide (the molar ratio of rhIL-1ra and PEG: 1/10; the molar mass of PEG: 5 kD) at 4° C. for 12 h. The reaction mixture was purified using HiTrap CM FF 5×1 ml (GE Healthcare). The column was washed, at the flow rate of 1 ml/min (Peristaltic Pump), with 5 column volumes of PBS and then 5 column volumes of buffer (pH 8.2/Phosphate/50 mM/Na⁺). The conjugate was eluted with the buffer. The eluates were analyzed to determine the amount of the conjugate bound to the column using a protein assay kit (BIO-RAD).

### (2) Synthesis of the Following IL-1ra Conjugate:

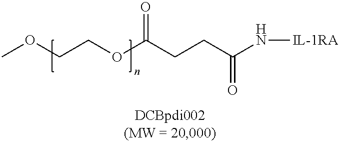

The conjugate was synthesized by the same method as described above, except that mPEG having a molecular weight of 30 kD, instead of mPEG having a molecular weight of 5 kD, was used.

### (3) Synthesis of the Following IL-1ra Conjugate:

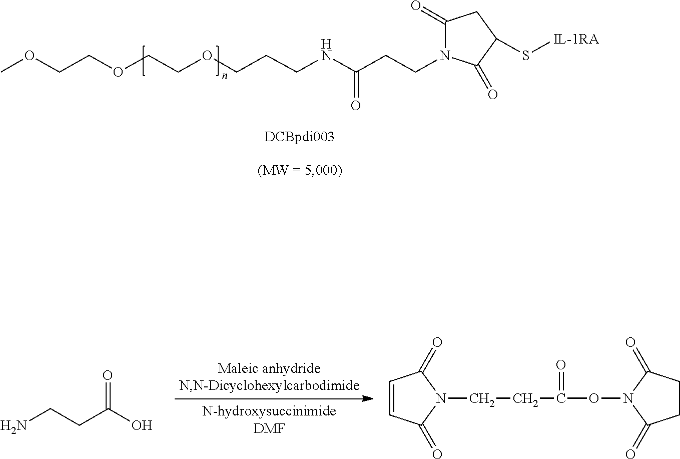

β-Alanine (1.00 eq.) was added to a solution of maleic anhydride (1.00 eq.) in dry dimethylformamide. The suspension was stirred for 1.0 h after the amino acid was dissolved. The resulting solution was cooled to 0° C. N-hydroxysuccinimide (1.25 eq.) was added followed by dicyclohexylcarbodiimide (2.00 eq.). After 5.0 min, the ice bath was removed and the solution was stirred for additional 18 h. The reaction mixture was extracted with dichloromethane and washed with water. The organic layer was dried over anhydrous sodium sulfate, concentrated under reduced pressure, and recrystallized in ether.

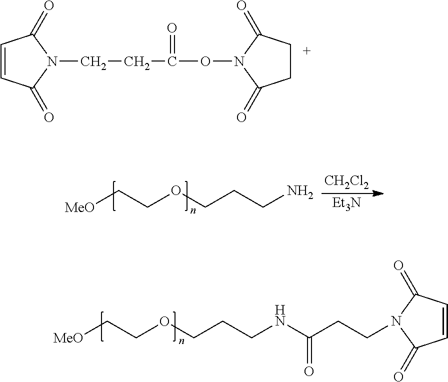

Succinimido 3-maleimidopropanoate (5.00 eq.) was dissolved in dry dichloromethane, followed by the addition of aminopropyl-mPEG (1.00 eq., MW=5000, purchased from NOF) and triethylamine (5.00 eq). The reaction was stirred at room temperature for 48 h. The solvent was removed and replaced with acetone. The solution was warmed to dissolve solids and then cooled to 0° C. White precipitate of polymer was obtained under vacuum.

RhIL-1ra (4 mg, 0.26 μmmol) at 1 mg/ml in PBS (pH 6.5) was conjugated with activated PEG (molar ratio of rhIL-1ra and PEG being 1/10) at 4° C. for 12 h. The reaction mixture containing PEG-IL-1ra was purified using HiTrap CM FF 5×1 ml (GE Healthcare). The column was washed, at the flow rate of 1 ml/min (Peristaltic Pump), with 5 column volumes of PBS and then 5 column volumes of a buffer (pH 4.3/acetic acid/50 mM/Na⁺). The conjugate was eluted with the buffer. The eluates were analyzed to determine the amount bound to the column using a protein assay kit (BIO-RAD).

### (4) Synthesis of the Following IL-1ra Conjugate:

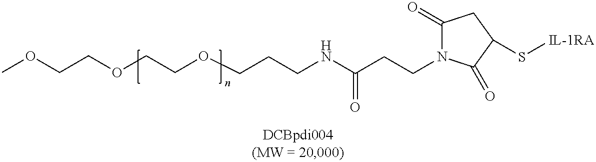

The conjugate was synthesized by the same method as described above except that mPEG having a molecular weight of 20 kD, instead mPEG having a molecular weight of 5 kD, was used.

### (5) Synthesis of the Following IL-1ra Conjugate:

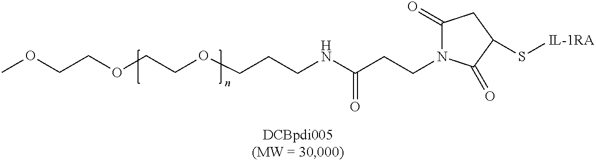

The conjugate was synthesized by the method described above except that mPEG having a molecular weight of 30 kD, instead mPEG having a molecular weight of 5 kD, was used.

### (6) Synthesis of the Following IL-1ra Conjugate:

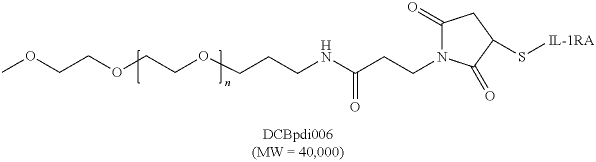

The conjugate was synthesized by the same method as described above except that mPEG having a molecular weight of 40 kD, instead mPEG having a molecular weight of 5 KD, was used.

### (7) Synthesis of the Following IL-1ra Conjugate:

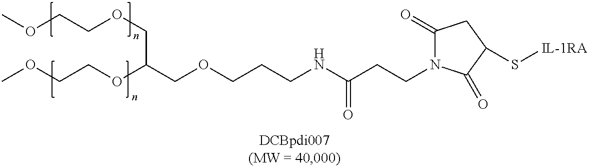

The conjugate was synthesized by the same method as described above except that di-branched mPEG having a molecular weight of 40 kD, instead mPEG having a molecular weight of 5 kD, was used.

### (8) Synthesis of the Following IL-1ra Conjugate:

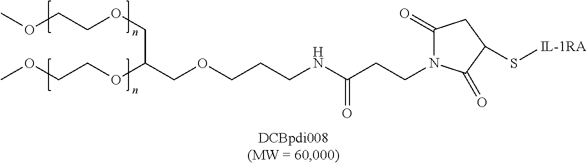

The conjugate was synthesized by the same method as described above except that di-branched mPEG having a molecular weight of 60 kD, instead mPEG having a molecular weight of 5 kD, was used.

### (9) Synthesis of the Following IL-1ra Conjugate:

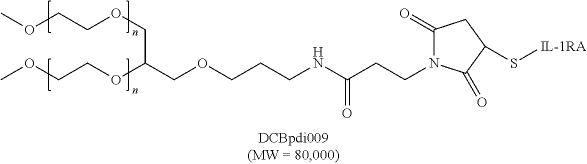

The conjugate was synthesized by the same method as described above except that di-branched mPEG having a molecular weight of 80 kD, instead mPEG having a molecular weight of 5 kD, was used.

### (10) Synthesis of the Following IL-1ra Conjugate:

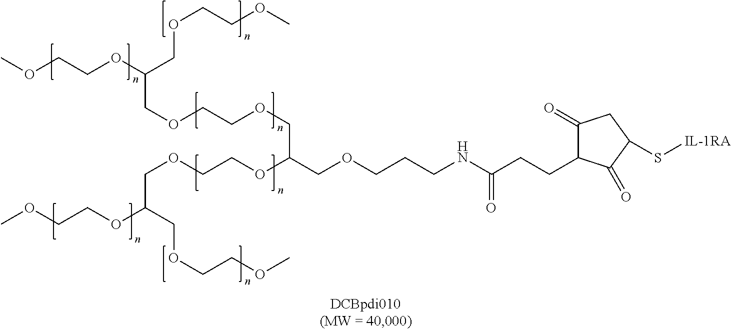

The conjugate was synthesized by the same method as described above except that tetra-branched mPEG having four a molecular weight of 40 kD, instead mPEG having a molecular weight of 5 kD, was used.

### (11) Synthesis of the Following IL-1ra Conjugate:

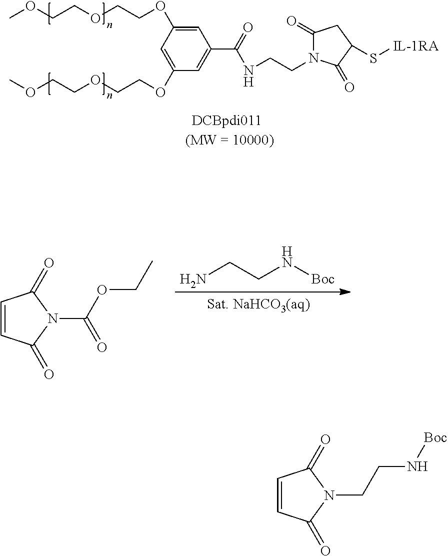

N-(ethoxycarbonyl) maleimide (0.53 g, 3.1 mmol) was added to N-(tert-butoxycarbonyl)-ethylenediamine (0.40 g, 2.5 mmol) in saturated aqueous bicarbonate solution (15 mL) at 0° C. The reaction mixture was stirred for 30 min at 0° C., and then stirred for an additional 1.0 hour at room temperature. The aqueous layer was extracted with methylene chloride (30 mL) three times. The combined organic layers were dried over anhydrous magnesium sulfate and concentrated under vacuum.

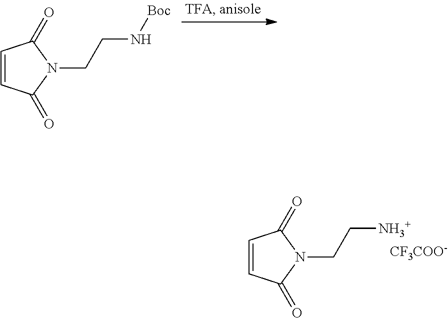

N-(2-((tert-Butoxycarbonyl)amino)ethyl)-maleimide (0.3 g, 1.25 mmol) in a solution of trifluoroacetic acid (4.0 mL) and anisole (0.15 mL, 1.39 mmol) was stirred for 1.0 h at room temperature. After trifluoroacetic acid was removed under vacuum, the residue was treated with dry ether to produce N-(2-aminoethyl)maleimide salt of trifluoroacetic acid as a white crystal.

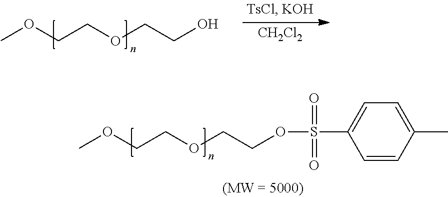

p-Toluenesulfonyl chloride (0.76 g, 4.0 mmol) was added to a solution of monomethoxypolyethylene glycol (MW=5000, 10.0 g, 2.00 mmol) in methylene chloride (40 mL) at 0° C. After the reaction mixture was stirred at 0° C. for 30 min, KOH (0.90 g, 16.0 mmol) was added. The reaction mixture was stirred at room temperature for 6.0 h. Then the mixture was filtered to remove KOH. The filtrate was extracted with methylene chloride and washed twice with water and brine. The organic layer was dried over MgSO₄ and filtered. The solvent was removed and replaced with acetone. The solution was warmed to dissolve solids and then ether was added at 0° C. White polymer precipitate was obtained under vacuum.

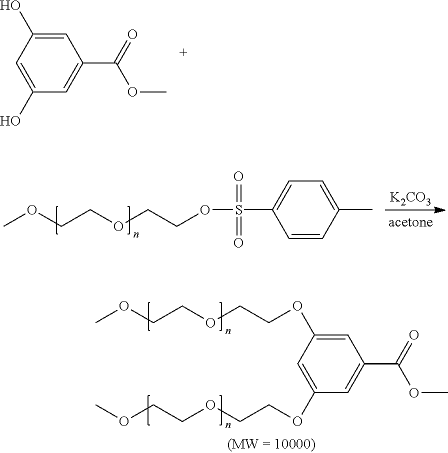

A solution of monomethoxypolyethylene glycol tosylate (MW=5000, 0.345 g, 0.07 mmol), methyl 3,5-dihydroxybenzoate (5 mg, 0.03 mmol), and K₂CO₃ (0.041 g, 0.3 mmol) in acetone (8 mL) was refluxed for 48 h. The mixture was filtered to remove K₂CO₃. The solvent was removed and replaced with acetone. The solution was warmed to dissolve solids and then ether was added at 0° C. White polymer precipitate was obtained under vacuum.

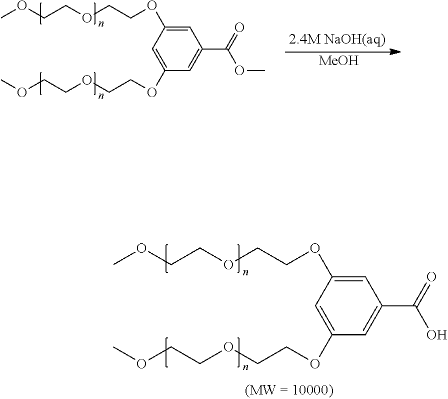

To a solution of methyl 3,5-bis-methoxypolyethylene glycol benzoate (0.25 g, 0.25 mmol) in MeOH (3 mL) was added 2.4 M NaOH (3 ml). The reaction mixture was stirred at room temperature for 48 h. MeOH was removed and acidified to pH 2 with 6.0 N HCl. The aqueous phase was extracted with methylene chloride (30 mL) three times. The organic layer was dried over MgSO₄ and filtered. The solvent was removed and replaced with acetone. The solution was warmed to dissolve solids and then ether was added at 0° C. White polymer precipitate was obtained under vacuum.

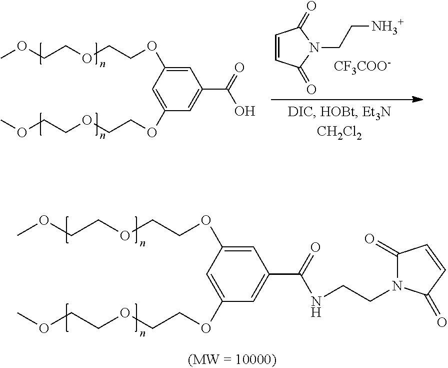

To a solution of 3,5-bis-methoxypolyethylene glycol benzoic acid (0.25 g, 0.02 mmol) and HOBt (0.027 g, 0.2 mmol) in CH₂Cl₂ (3 mL) were added N,N′-diisopropylcarbodiimide (0.031 mL, 0.2 mmol) and N-(2-Aminoethyl)maleimide salt of trifluoroacetic acid (0.015 g, 0.06 mmol). Then, triethylamine (0.15 mL) was added and the reaction mixture was stirred at room temperature for 48 h. The mixture was extracted with methylene chloride and washed twice with water and brine. The organic layer was dried over MgSO₄ and filtered. The solvent was removed and replaced with acetone. The solution was warmed to dissolve solids and then ether was added at 0° C. Pink polymer precipitate was obtained under vacuum. Di-branched mPEG-maleimide was purified by gel filtration chromatography using a bio Gel P100(Bio-red) column (1.6×80 cm) and water as eluent.

### (12) Synthesis of the Following IL-1ra Conjugate:

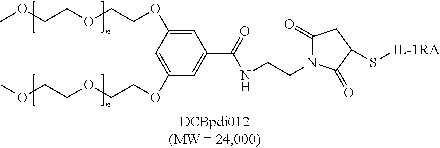

The conjugate was synthesized by the same method as described above except that di-branched mPEG having a molecular weight of 24 kD (each mPEG branch having a molecular weight of 12 kD), instead of mPEG having a molecular weight of 10 kD, was used.

### (13) Synthesis of the Following IL-1ra Conjugate:

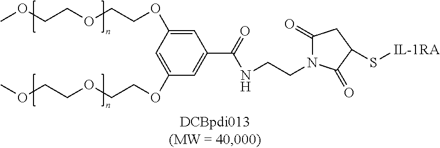

The conjugate was synthesized by the same method as described above except that di-branched mPEG having a molecular weight of 40 kD (each mPEG branch having a molecular weight of 20 kD), instead of mPEG having a molecular weight of 10 kD, was used.

(14) SEC-HPLC Analysis of Di-Branched m-PEGs:

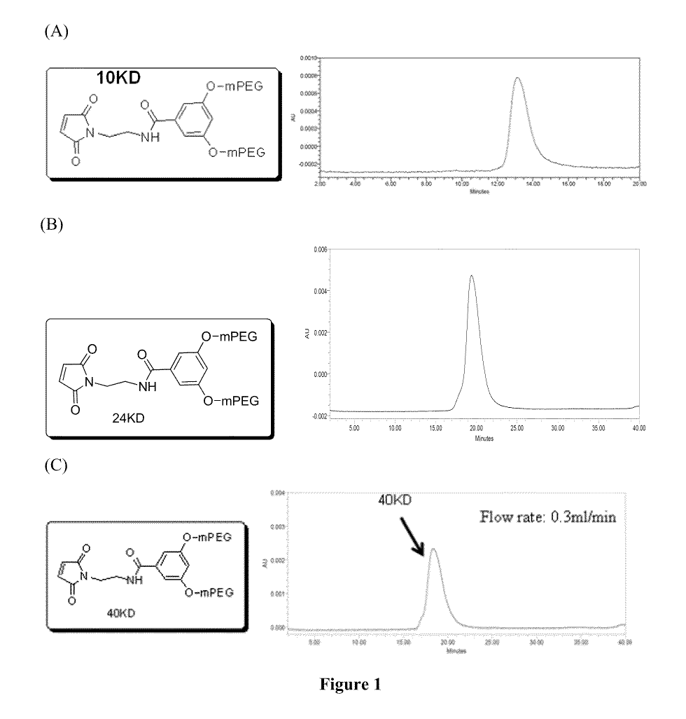

Purities of dibranched mPEGs used to prepare Conjugates 11, 12, and 13 were detected using SEC-HPLC. The flow rate for m-PEG (WM=10 kD) was 0.5 mL/min and the flow rate for m-PEGs (MWs=24 and 40 kD) was 0.3 mL/min. As shown in FIGS. 1(A)-(C), these three mPEGs were all pure.

### (15) SEC-HPLC Analysis of PEG-IL-1ra Conjugate:

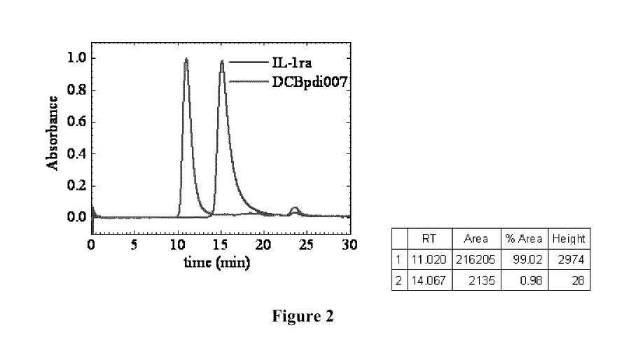

DCBpdi007 was purified by ion exchange column and size exclusion column. Its purity was detected using a SEC-HPLC system equipped with Waters 600 controller, Waters 717 plus autosampler, Waters 486 tunable absorbance detector, and Waters empower pro. The Waters Ultrahydrogel 250 (7.8×300 mm) was run under the isocratic condition at the flow rate of 0.4 mL/min or 0.5 mL/min using 0.1 M sodium nitrate or 1×PBS as the eluent. The sample was diluted to 0.2 mg/mL. Ten microliters of the diluted sample was injected and detected at 280 nm. As shown in FIG. 2, DCBpdi007 had a purity of 99%.

### (16) Quantification of Protein (PEG-IL-1Ra)

Protein or PEGylated proteins were quantified using bicinchoninic acid protein assay kits following the protocol recommended by manufacturer (Thermo Scientific).

### Example 2

### Biological Assays

### Material and Methods

### (1) IL-1RI Binding Assay

A coating buffer containing IL-1RI at concentration of 1 ug/ml was coated on plates (100 μl/well). The plates were sealed and stored at 4° C. overnight until use. The coating buffer was then aspirated and the wells washed 3 times with 300 μl/well of PBS. The wells were then incubated with a blocking buffer (PBS containing 1% BSA, 300 μd/well). After the plate was incubated at 37° C. for 2 hours, the blocking buffer was removed and wells washed 3 times with 300 μl/well PBST (0.05% Tween20). Samples were added (100 μl/well) by 2 folds serial dilution with PBS-1% BSA to the wells. The plates were sealed and incubated at 37° C. for 2 hours. The wells were washed in the same manner described above 3 times with 400 μl/well of PBST (0.05% Tween20). Anti-IL-1ra-biotin (1:300) in PBS-1% BSA was then add to each well (100 μl/well) before the plate was sealed and incubated at 37° C. for 2 hours. After washing the wells 6 times with 300 μl/well of PBST (0.05% Tween20), Steptavidin-HRP (1:4000) in PBS-1% BSA was add to each well (100 μl/well) and incubated at 37° C. for 2 hour. Then, TMB was added to each well (100 μl/well) and incubated at room temperature for 5-10 minutes for color development. The color development was stopped by adding 100 μl of 1N HCl. A microplate reader was then used to read the plates and obtain absorbance at 450-655.

### (2) Stability Assay In Vitro

Stability Assay In Vitro

To measure plasma concentration, IL-1ra and PEG-IL-1ra were incubated in human serum. Plasma samples were collected at different time points up to 24 (or 72) hours and their IL-1ra or PEG-IL-1ra concentrations were measured by Enzyme-linked immunosorbent assay (ELISA) in the manner described above.

Protease Inhibitor Assay

IL-1ra was incubated in human serum in the presence of or the absence of protease inhibitor (Halt™ protease inhibitor cocktail kit, PIERCE) at 37° C. for 0, 2, 4, 8, or 24 hours. Serum samples were collected at different time points and the concentration of IL-1ra was measured by ELSIA in the manner described above. Captured antibody was transferred to an ELISA plate (100 μl/well, diluted to 2 ug/ml concentration in PBS) and incubated overnight at room temperature. Each well was then washed with a wash buffer (3000) three times, blocked with 300 μl of PBS containing 1% BSA at room temperature for 1 hour. After each well was washed again in the same manner, 1000 of samples or IL-1ra standards in an appropriate diluent was added to the wells and incubated for 2 hours at room temperature. After washing three times, biotinylated detection antibody (diluted in the same diluent with 1% BSA) was added to each well (100 μl/well) and incubated for 2 hours at room temperature. After washing again, each well was incubated with 100 μl Streptavidin HRP for 1 hour at room temperature. The color development was conducted using 100 μl of a substrate solution for 30 minutes at room temperature before it was stopped by incubating with 100 μl of 1N HCL. The optical density (O.D.) 450-655 nm of each well was determined within 30 minutes using a microplate reader in the same manner described above.

### (3) Cell Proliferation Assay of IL-1

Serial dilutions of human IL-1 beta (R&D) in duplicates were mixed with fixed number of D10 cells in a 5% T-STIM ConA, 10% FBS, RPMI-1640 medium supplemented with L-glutamine and 2ME in a 96-well assay plate in a total volume of 200 μl/well. The background wells with cells in the medium only were also included for assay. The assay plate was incubated in a humidified chamber at 37° C. and 5% CO₂ incubator for 3 days. MTS assay solution (PROMEGA) was added into each well of the assay plate at the end of incubation. The assay plate was incubated for additional 4-5 hours for the color development. The O.D. of each well of the assay plate, which is directly proportional to the total number of living cells in the well, was read in a plate reader at 490-655 nm. Cell proliferation curve was plotted with O.D. vs. IL-1 concentration (ng/ml).

### (4) IL-1 Neutralization Assay of Receptor Antagonist

Serial dilutions of IL-1 receptor antagonists (IL-1ra and PEG-IL-1ra) in duplicates were mixed with fixed number of D10 cells in a 5% FBS, RPMI-1640 medium supplemented with L-glutamine and 2ME in a 96-well assay plate. The assay plate was pre-incubated for 1 hour at 37° C. Human IL-1 alpha at fixed concentration was added into each well of the assay plate so the final concentration of hIL-1a was 1 ng/ml. Control wells with cells and hIL-1a (1 ng/ml) only were also included for assay. The assay plate with total volume of 200 μl/well was incubated in humidified chamber at 37° C. and 5% CO₂ incubator for 3 days. MTS (PROMEGA) assay was conducted and analyzed as described above. The neutralization curves were plotted with O.D. vs. the concentrations of receptor antagonist (ng/ml).

### (5) In Vivo Pharmacokinetics of PEG-IL-1ra

Sprague-Dawley (SD) male rats (approximately 300-350 g each) were obtained from BioLASCO (Taipei, Taiwan). The rats were individually housed and fed a Laboratory Autoclavable Rodent Diet (PMI®, Nutrition International, Inc., MO., USA) through out the study period. All in vivo studies were approved by IACUS animal study protocol.

Before dosing, all animals were weighted and observed for clinical signs. Any animals showed sign of illness were removed from study. A final of 16 animals were randomly allocated into four groups, based on their weight classes. Dosing level used 3 mg/kg in this study. A total of four test articles, KINERET, DCBpdi005, DCBpdi006, and DCBpdi007, were used. The dosing level, volume and concentration used were indicated in Table I. All test articles were administrated via intravenous route (iv).

Blood samples (approximately 0.25 ml/animal) were collected from tail vein before dosing and at 15 minutes, 1, 2, 4, 6, 8, 24 and 32 hours after iv administration. Blood samples (about 0.5 ml/animal) were collected from the tail vein at 48, 72, 96 and 120 hours after doing. On the sixth days post dosing (144 hours), blood was fully drawn from all animals. All blood samples were kept on ice or maintained under 4° C. with EDTA as anticoagulant. To obtain plasma, blood samples were centrifuged at 1000 G for 15 minutes at 4° C. Plasma samples were stored in a −80° C. freezer prior to analysis.

TABLE I

| | | Dosing | | Dosing | |
|---|---|---|---|---|---|
| Group | | Level | Concentration | Volume | Numbers of |
| Number | Test Article | (mg/kg) | (mg/ml) | (ml/kg) | Animal |
| 1 | KINERET | 3 | 1 | 3 | 4 |
| 2 | DCBpdi005 | 3 | 1 | 3 | 4 |
| 3 | DCBpdi006 | 3 | 1 | 3 | 4 |
| 4 | DCBpdi007 | 3 | 1 | 3 | 4 |

### Results

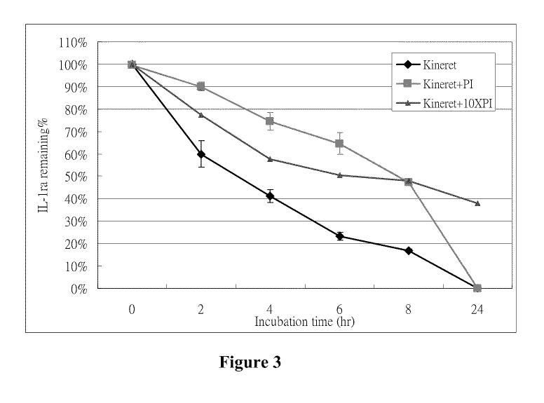

It was found that IL-1ra had a poor stability in serum which was caused by protease degradation. As mentioned above, IL-1ra was incubated with or without a protease inhibitor in human serum samples at 37° C. for 0, 2, 4, 8, 24 hours. The samples were collected at different time points up to 24 h and the concentration of IL-1ra was measured by ELISA. It was found that the half life of IL1-ra in human serum was about 4 hours (FIG. 3). It was also found that protease inhibitor cocktails at different concentration (1× or 10×, PIERCE) could increase the stability of IL1-ra in human serum. This result demonstrated that IL-1ra was rapidly degraded by, at least partially, proteolysis in blood.

To increase the serum stability of IL-1ra, an Fc molecule was used to the C-terminus of IL-1ra to increase the molecule size. The result indicated that increasing the molecule size did not increase the half-life of IL-1ra in serum circulation. Thus, it is not sufficient only to increase the molecule size, and the issue of protease digestion has to be addressed.

One way to increase the serum half life of IL-1ra is to prevent or decrease the proteolysis process on IL-1ra. Poly(ethylene glycol) (PEG) chains conjugated to therapeutic peptides and proteins play a critical role in preventing proteolytic degradation by various proteases present in blood and tissues. To create proteins with IL-1ra activity and prolonged half-life, various kinds of PEG-IL-1ra with different molecular weights were generated.

More specifically, a series of PEGylated IL-1ra were designed. First, mPEG-succinyl-NHS was attached to IL-1ra at lysine residues to generate 2 PEGylated IL-1ra proteins with different molecular weights. Examples included DCBpdi001 (MW=5,000) and DCBpdi002 (MW=20,000) (Table 1). Second, PEGylate IL-1ra was designed to have PEG molecules attached at cysteine residues with different molecular weights. Different types of PEG molecules were used to synthesize these PEG-IL-1ra proteins. They included linear mPEG-maleimide (e.g., DCBpdi003 (MW=5,000), DCBpdi004 (MW=20,000), and DCBpdi005 (MW=30,000); DCBpdi006 (MW=40,000)); 2 branched mPEG-maleimide (e.g., DCBpdi007 (MW=40,000); DCBpdi008 (MW=60,000); DCBpdi009 (MW=80,000); DCBpdi011 (MW=10,000); DCBpdi012 (MW=24,000); DCBpdi013 (MW=40,000)), and 4 branched mPEG-maleimide (e.g., DCBpdi010 (MW=40,000)).

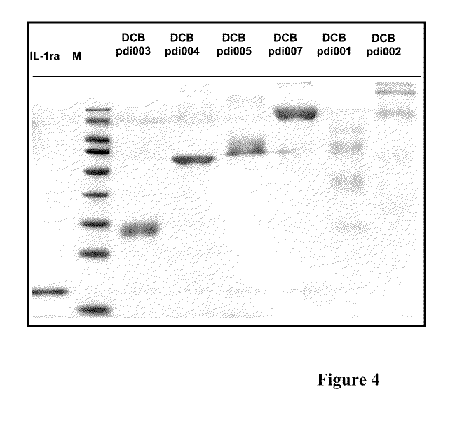

IL-1ra and PEG were conjugated in conditioned buffer (PBS, pH6.5/pH7.5) at 4° C. for 12 hrs and PEG-IL-1ra was purified from conditioned buffer by ion exchange HiTrap column (GE Healthcare). PEGylated IL-1ra protein concentrations were determined by BCA protein assay and analyzed by SDS-PAGE (FIG. 4). The various forms of the PEG-IL-1ra were examined to determine their ability of binding to recombinant soluble human IL-1 Type I receptor (IL-1RI) using ELISA. The IL-1 Type I receptor (IL-1RI) complex appears to mediate all known IL-1 biological responses. Formation of the IL 1-RI complex with its ligands was the first step to trigger all the ensuing biological responses.

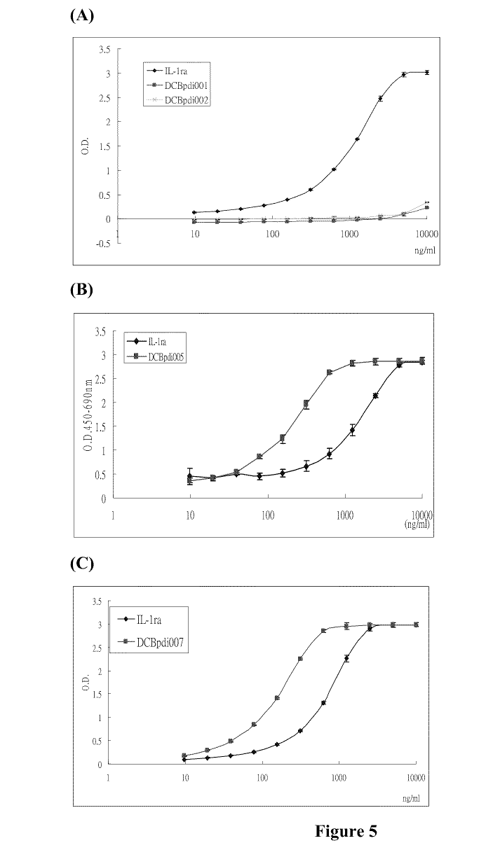

It was found that both IL-1ra proteins and unselectively PEGylated on lysine residues (DCBpdi001 and DCBpdi002), completely lost their binding activity to IL1-RI (FIGS. 4 and 5, Table 2).

TABLE 2

| | Binding affinity | | |
|---|---|---|---|
| | Kd₅₀ improved as | Stability | Neutralization activity |
| PEGylated | fold compared to | IL-1RA | EC₅₀ ratio |
| IL-1ra | Kd₅ of IL-1ra | (t_{1/2}, hr) | (=PEG-IL-1RA/IL-1RA) |
| DCBpdi001 | Undetectable | 3 | NA |
| DCBpdi002 | Undetectable | 3.8 | NA |
| DCBpdi003 | 5.2 | 3.85 | 7.4 |
| DCBpdi004 | 8.6 | 7.28 | 20.9 |
| DCBpdi005 | 3.24 | >72 | 12.2 |
| DCBpdi006 | 1.36 | >72 | 26.8 |
| DCBpdi007 | 1.45 | >72 | 28.3 |
| DCBpdi008 | 1.03 | >72 | NA |
| DCBpdi009 | 0.39 | >72 | NA |
| DCBpdi010 | >1 | 3.2 | 39 |
| DCBpdi012 | 1.33 | >72 | NA |
| DCBpdi013 | 1.51 | >72 | NA |
| IL-1ra | 1.0* | 3.5 | — |

NA: not available
*ratio 1.0 corresponds to a binding affinity/Kd₅₀ of 1070 ± 320 ng/ml

As shown in Table 2, when IL-1ra proteins were conjugated with PEG whose molecular weight is below 60 kD (e.g., DCBpdi003-008, 010, 012-013), they exhibited binding activities to IL-1RI that were comparable to, or better than, that of native IL-1ra. (FIGS. 5B and 5C).

To evaluate the stability of PEG-IL-1ra in human serum, the above-mentioned IL-1ra PEG conjugates were incubated with human serum for up to 72 hours and their concentrations were measured at various time points by ELISA. The results were shown in Table 2 above.

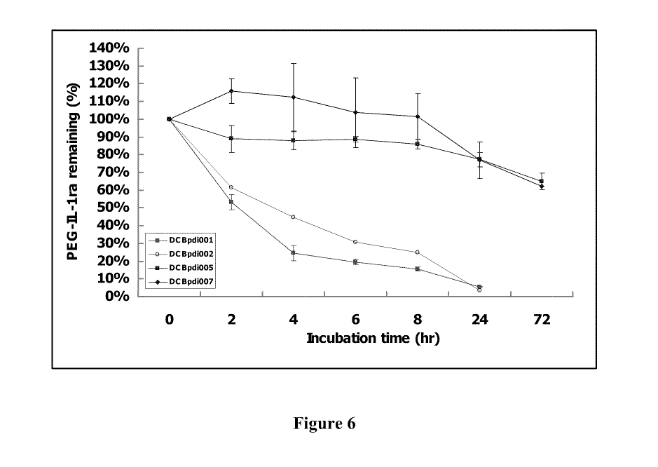

It was found that, similar to native IL-1ra, the two Lys-PEGylated IL-1ra, i.e., DCBpdi001 and DCBpdi002, quickly degraded and became undetectable within 24 hrs (FIG. 6). In contrast, DCBpdi005-09 and 012-013, in which PEF were conjugated at Cys-residues and had molecular weights above 20 kD, appeared to be more resistant to proteolysis degradation in human serum than native IL-1ra. As shown in Table 2, the half-life (t_{1/2}) of these seven conjugates exceeded 72 hr in human serum (Table 2). DCBpdi010, which had 4-branched PEG (each chain of which was less than 10 kD) had a half-life similar to that of native IL-1ra.

As the activity of DCBpdi005 and DCBpdi007 to bind to IL1-RI was unaffected by PEGylation, the biological activity of DCBpdi005 and DCBpdi007 was examined using IL-1ra neutralization assay.

In brief, the biological activity of each of the two PEG-IL-1ras was measured by its capability to inhibit cell proliferation of a murine helper T cell line, D10.G4.1, of which growth is IL-1 dependent. IL-1ra was able to compete with IL-1 for binding to cell surface IL-1 receptor with high affinity and block the IL-1 induced cell proliferation.

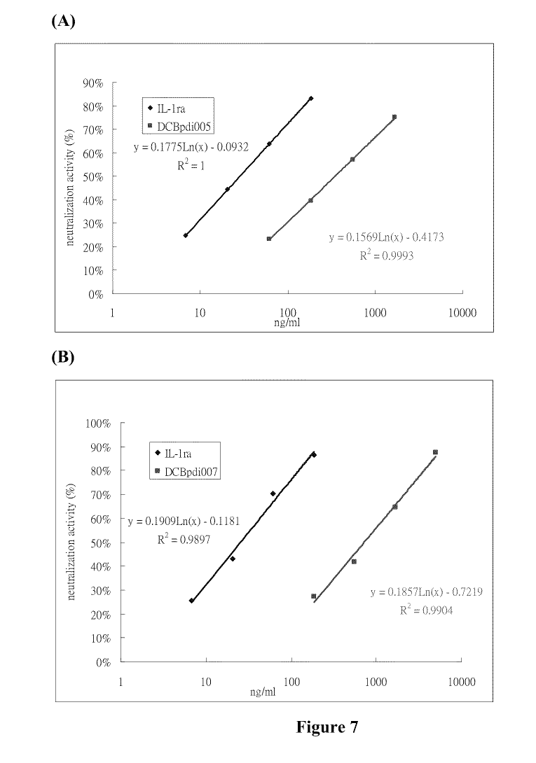

It was found that selective PEGylation at Cys-residues appeared to decrease the neutralization capability of IL-1ra for IL-1β which, however, only partially affected this biological activity of IL-1ra (Table 2). The EC₅₀ value of IL-1ra is 27 ng/ml while those of DCBpdi005 and DCBpdi007 are 346 ng/ml and 720 ng/ml, respectively (FIG. 7). The neutralization activities of Cys-selective PEG-IL-1ra proteins, DCBpdi005 and DCBpdi007, were about 10-30 folds less than IL-1ra (Table 2).

Therapeutic uses of IL-1ra in human are greatly limited since it is quickly cleared from serum circulation due to its relative small size and susceptibility to proteolysis degradation. The inherent nature of IL-1ra leads to the development of ANAKINRA with a daily dosing regimen, which inevitably leads to unfavorable side effects. The small size of IL-1ra could be overcome by conjugation of different sizes of PEG. However, it is difficult for PEGylation to decrease or prevent proteolysis degradation of IL-1ra in serum without sacrificing its binding and neutralizing activity.

Many studies attempted but failed to improve the stability of IL-1ra in serum to a favorable level for medical use so far. For example, none of the studies could successfully extend the serum half-life up to 24 hr. The above described DCBpdi005-008 and DCBpdi012-013 are the first examples that have human serum half-lives of more than 72 hrs, while remain biological potent to bind IL1-RI (FIGS. 5-7 and Table 2). All of these PEG-IL-1ra conjugates had mPEG-maleimide (24 to 60 kD) conjugated at Cys-residues.

The above results demonstrate that both size of PEG and sites of PEGylation are crucial to improve stability and retain biological activity of IL-1ra in human serum. The size of PEG for conjugation, linear or branched, preferable should exceed 20 kD so as to efficiently inhibit protease digestion of IL-1ra but lower than 60 kD to remain the binding ability to IL-1RI.

In addition to size, the PEGylation sites are also important. PEGylation on cysteine residues (Cys 66, Cys 69, Cys 116, or Cys 122) plays a crucial role for the stability and activity of IL-1ra. Cysteine residues potential to form intra-molecular bonds are traditionally considered important for protein structure and thus biological activity. Formation of an intra-molecular bonding between Cys 69 and Cys 116 was proposed although some evidences suggested otherwise. It was unexpected that chemical modification on cysteine by PEFGylation, at least with some forms of PEG, does not abolish the binding and biological activity of IL-1ra.

Finally, the above result also demonstrated that different forms of PEG were important for the stability and biological activity of IL-1ra, which is evidenced by the observation that linear and 2-branched mPEG-maleimide but not 4-branched mPEG-maleimide could improve the stability of IL-1ra (Table 2).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A conjugate of Formula (I): 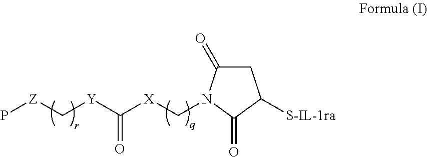
wherein
each of X, Y, and Z, independently, is O, NH, or deleted;
S-IL-1ra is IL-1ra protein, a sulfur atom of which is linked to the succinimidyl ring in Formula (I);
P is a linear or branched polymeric moiety; and
each of r and q, independently, is 0, 1, 2, 3, 4, or 5.

2. The conjugate of claim 1, wherein P has a molar mass of 5-100 kD.

3. The conjugate of claim 2, wherein P is a linear polyethylene glycol moiety.

4. The conjugate of claim 3, wherein P has a molar mass of 20-60 kD.

5. The conjugate of claim 2, wherein P is a branched polyethylene glycol moiety.

6. The conjugate of claim 5, wherein P has the following structure: 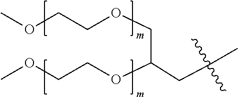
wherein each m, independently, is 250-1000.

7. The conjugate of claim 5, wherein P has the following structure: 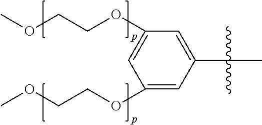
wherein each p, independently, is 250-700.

8. The conjugate of claim 1, wherein q is 2.

9. The conjugate of claim 8, wherein X is deleted and Y is O or NH.

10. The conjugate of claim 9, wherein r is 3 and Z is deleted.

11. The conjugate of claim 10, wherein P is a linear polyethylene glycol moiety.

12. The conjugate of claim 11, wherein P has a molar mass of 5-40 kD.

13. The conjugate of claim 9, wherein r is 3 and Z is O.

14. The conjugate of claim 13, wherein P has the following structure: 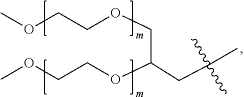
wherein each m, independently, is 250-1000.

15. The conjugate of claim 8, wherein X is O or NH and Y is deleted.

16. The conjugate of claim 15, wherein P is 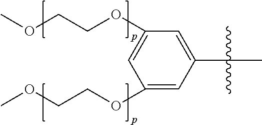
wherein each p, independently, is 250-700.

17. The conjugate of claim 1, wherein the conjugate has the following formula: 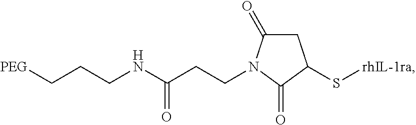
in which the PEG moiety has a molar mass of about 30 kD.

18. The conjugate of claim 1, wherein the conjugate has the following formula: 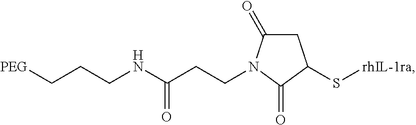
in which the PEG moiety has a molar mass of about 40 kD.

19. The conjugate of claim 1, wherein the conjugate has the following formula: 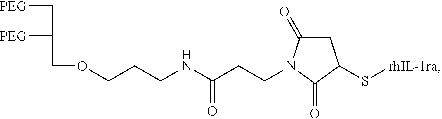
wherein each of the PEG moieties has a molar mass of about 20 kD.

20. The conjugate of claim 1, wherein the conjugate has the following formula: 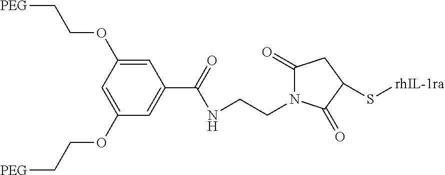
wherein each of the PEG moieties has a molar mass of about 12 kD.

21. The conjugate of claim 1, wherein the conjugate has the following formula: 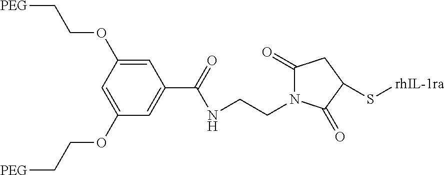
wherein each of the PEG moieties has a molar mass of about 20 kD.

22. The conjugate of claim 1, wherein the conjugate has a half-life in human serum of more than 12 hours.

23. The conjugate of claim 22, wherein the conjugate has a half-life in human serum of more than 48 hours.

24. The conjugate of claim 23, wherein the conjugate has a half-life in human serum of more than 72 hours.

25. A pharmaceutical composition comprising a pharmaceutically acceptable carrier and the conjugate of claim 1.

26. A method of treating an immune disorder in a subject, comprising administering to the subject in need thereof an effective amount of the conjugate of claim 1.

27. The method of claim 26, wherein the immune disorder is rheumatoid arthritis.


SEQUENCE LISTING (1 sequence)

SEQ ID NO: 1 - protein, 153 aa, Homo sapiens
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE